(19) United States
(12) Patent Application Publication
(10) Pub. No.: US 2026/0233009 A1
(43) Pub. Date: Aug. 13, 2026

(54) Personalized Neuromodulation Procedure via Machine Learning

(71) Applicants: Georgia State University Research Foundation, Inc., Atlanta, GA (US); Emory University, Atlanta, GA (US); Georgia Tech Research Corporation, Atlanta, GA (US)

(72) Inventors: Mohammad Sadegh Eslampanah Sendi, Atlanta, GA (US); Vince Calhoun, Atlanta, GA (US); Robert Gross, Atlanta, GA (US)

(21) Appl. No.: 19/474,111

(22) PCT Filed: Apr. 10, 2024
(86) PCT No.: PCT/US2024/023840

§ 371 Date: Oct. 9, 2025

Related U.S. Application Data
(60) Provisional application No. 63/495,368, filed on Apr. 11, 2023.

(51) Int. Cl. A61N 1/36

(52) U.S. Cl. CPC A61N 1/36139; A61N 1/36189

(57) ABSTRACT

A method for treating a patient comprises the steps of: receiving a set of information describing the patient, identifying a biomarker associated with a condition affecting the patient, determining a functional link between the biomarker and at least one modulation parameter by using an active learning framework, and applying the functional link to treat the condition in the patient by modulating at least one specific region of the patient.

### RELATED APPLICATIONS

This application claims the benefit of and priority to U.S. Provisional Patent Application No. 63/495,368 filed Apr. 11, 2023, the contents of which are incorporated herein by reference in their entirety.

### STATEMENT REGARDING FEDERALLY SPONSORED RESEARCH

This invention was made with government support under contracts MH123610 and NS100559 awarded by the National Institutes of Health. The government has certain rights in the invention.

### BACKGROUND

The medical community constantly seeks improvements for treating health conditions, such as injuries, disorders and other physical or psychological conditions or traumas. In one example, neurological disorders comprise more than 600 conditions that impact an estimated 50 million Americans every year. This type of disorder causes impairment in the central nervous system's functionality or the peripheral nervous systems and chronic physical, cognitive, and emotional disability. Continuing with the example, brain stimulation, the focused delivery of current (usually a square pulse) to the brain to affect neural or physiological processes, is a common method of studying the physiology of the nervous system. It is also successfully used to treat conditions, or disorders, in which the nervous system is affected or implicated. Similar modulation therapies may be used to treat other conditions. To optimize treatment parameters for individual patients, it is essential to comprehend the organ's response as a complex system to various treatment parameter sets. This necessitates establishing a model that correlates treatment parameters with organ response, as indicated by target biomarkers. However, modeling this relationship requires numerous samples, which is often expensive, challenging, and impractical in clinical settings. Therefore, an optimal experimental design is crucial for identifying the most informative samples to elucidate this connection, both in clinical and experimental environments. Previous systems use random selection or passive learning techniques. In a classical (or random) learning approach, this process typically needs a large amount of training data. In a passive learning approach, in which data is collected through a random sampling (RS) process, a learner uses each observation obtained from each sample to update the model parameters. However, selecting appropriate control variables for improving the learning process may not be considered. Therefore, a large amount of data may be difficult to obtain, particularly when expensive and time-consuming clinical experiments are needed. Also, outcomes can vary from patient to patient and may not be predictable. Improvements are needed.

Accordingly, a need arises for improvements in identifying and treating health conditions.

### SUMMARY

The present disclosure is directed, at least in part, to modulation methods responsive to biomarker identification. In some specific implementations, the methods relate to methods of modulation and systems for executing modulation methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a method for treating a patient including the steps of: receiving a set of information describing the patient, identifying a biomarker associated with a condition affecting the patient, determining a functional link between the biomarker and at least one modulation parameter by using an active learning framework, and applying the functional link to treat the condition in the patient by modulating at least one specific region of the patient. Other embodiments of this aspect include corresponding computer systems, apparatus, and computer programs recorded on one or more computer storage devices, each configured to perform the actions of the methods.

One general aspect includes a system for treating a patient may include a processor and memory, wherein computer instructions are stored in the memory to cause the processor to perform the steps of: receiving a set of information describing the patient, identifying a biomarker associated with a condition affecting the patient, determining a functional link between the biomarker and at least one modulation parameter by using an active learning framework, and applying the functional link to treat the condition in the patient by modulating at least one specific region of the patient. Other embodiments of this aspect include corresponding computer systems, apparatus, and computer programs recorded on one or more computer storage devices, each configured to perform the actions of the methods.

One general aspect includes a computer-readable storage medium storing instructions, an execution of which in a computer system causes the computer system to perform operations comprising: receiving a set of information describing the patient, identifying a biomarker associated with a condition affecting the patient, determining a functional link between the biomarker and at least one modulation parameter by using an active learning framework, and applying the functional link to treat the condition in the patient by modulating at least one specific region of the patient. Other embodiments of this aspect include corresponding computer systems, apparatus, and computer programs recorded on one or more computer storage devices, each configured to perform the actions of the methods.

### BRIEF DESCRIPTION OF THE DRAWINGS

So that the manner in which the above recited features of the present disclosure can be understood in detail, a more particular description of the invention, briefly summarized above, may be had by reference to embodiments, some of which are illustrated in the appended drawings. It is to be noted, however, that the appended drawings illustrate only typical embodiments of this invention and the invention may admit to other equally effective embodiments.

FIG. 1 illustrates a flow diagram of a method 100 for treating a patient.

FIGS. 2A-2B illustrate block diagrams of a system for identifying a response biomarker.

FIGS. 3A-3B illustrate an example of biomarker identification.

FIGS. 4A-4B illustrate experimental results from a classification.

FIGS. 5A-5B illustrate predicting the memory performance in a one-day test phase based on the CAI and the amygdala activities during the study phase.

FIG. 6 illustrates the proposed active learning (AL) method.

FIG. 7 illustrates an active learning model outperforming the random sampling model.

FIG. 8 is a summary plot of models' performance on the Parkinson's disease parameter sweep data.

FIG. 9 illustrates an active learning approach outperforming the random sampling approach.

FIG. 10 illustrates performance of the model on the medial septum optogenetic parameter sweep data.

FIG. 11 illustrates AL vs. RS in a real-time in vivo medial septum optogenetic experiment.

FIG. 12 illustrates a block diagram of a computing device.

Other features of the present embodiments will be apparent from the Detailed Description that follows.

### DETAILED DESCRIPTION

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. Electrical, mechanical, logical, and structural changes may be made to the embodiments without departing from the spirit and scope of the present teachings. The following detailed description is therefore not to be taken in a limiting sense, and the scope of the present disclosure is defined by the appended claims and their equivalents.

The present disclosure relates to modulation techniques for treating health conditions such as physical ailments, mental illnesses or other conditions, disorders, injuries or traumas. Modulation methods may include pharmacological and non-pharmacological modulation methods In one example, neuromodulation therapies are disclosed, where neuromodulation is defined as a neurosurgical treatment that modulates brain neural functioning by delivering an electrical signal using predefined stimulation parameters to a specific deep anatomical structure of the central nervous system. The patient-specific (or personalized) modulation therapies methods and systems disclosed herein result in reduced patient variability and increased reproducibility of results or treatments.

To accomplish personalized modulation, a new procedure for personalized modulation for any individual through a machine learning approach is disclosed. Upon receiving inputs relating to a patient and one or more biomarkers indicating a condition affecting the patient, a link between a treatment parameter and a response is identified. To build such a link, a model may be trained from available data and may be used to predict the response for untested modulation parameters. An ultimate goal of personalized modulation disclosed herein is to increase the success rate of modulation therapy and reduce the cost of these types of therapy.

Previous studies used Bayesian optimization (BaO) to optimize the experimental procedure in neuromodulation. There are a few fundamental differences between the framework disclosed herein and previous work in, for example, neuromodulation. First, the objective of previous methods was to select the best stimulation parameter and collect the most informative observation to maximize or minimize the brain feature (or biomarker) while minimizing the number of trials. An objective of the disclosed approach is to find a best regression model (or map) between one or more stimulation parameters and a feature associated with a physical or neurological characteristic, also referred to herein as a biomarker, with a minimum number of trials. Once a best regression model is obtained, a response (e.g., a brain response) to an untested stimulation parameter may be predicted. Therefore, the model may be used to minimize or maximize the target biomarker by finding the best stimulation parameters. Second, previous methods, such as Bayesian optimization, are bounded to Gaussian process regression (GPR), while, in the methods disclosed herein, any kind of regression model may be utilized. GPR might not provide the best model for predicting responses from the stimulation parameters. Therefore, the methods disclosed herein may outperform previous approaches by providing an improved regression model.

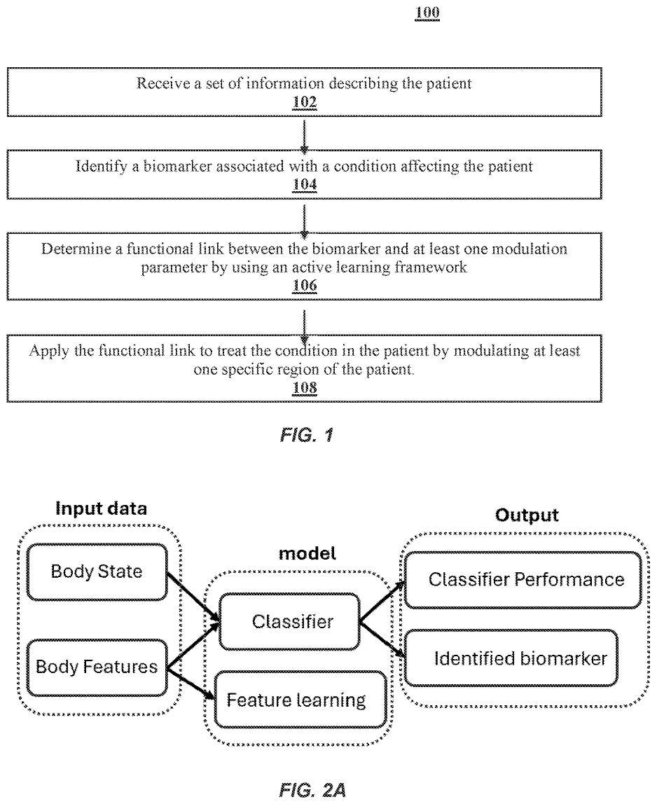

FIG. 1 illustrates a flow diagram of a method 100 for treating a patient. The method 100 comprises receiving 102 a set of information describing the patient. The receiving includes identifying a biomarker associated with a condition of a patient. As defined herein, a condition may refer to any health condition affecting a patient, such as any mental, neurological, psychological, or physiological ailment, injury, disease, disorder, condition, or trauma. Biomarker identification may be accomplished via interpretable artificial intelligence methods for identifying biomarkers associated with a corporal response to modulation. As defined herein, a corporal response may be any response to modulation of any body part, organ, or region, or the body as a whole. As defined herein, modulation can be any stimulation or manipulation of any area of the body, or any input into the body or a region of the body. Within this step, an intelligent, data-driven approach for identifying the biomarkers of the effects of modulation is developed. Statistical hypothesis testing methods do not account for the joint space interactions of markers associated with activity, and cannot indicate the likelihood of inter-subject generalizability of identified biomarkers. An interpretable machine learning classification approach may be utilized to investigate the effects of corporal modulation in target body areas and identify the biomarkers that differentiate various modulatory states.

The method 100 further comprises identifying 104 a biomarker associated with a condition affecting the patient. Identifying a biomarker includes establishing an intelligent, data-driven approach for identifying the biomarkers of modulation effects. An interpretable machine learning classification approach may be utilized to investigate the effects of stimulation in body areas, and identify the features or biomarkers that differentiate various corporal states.

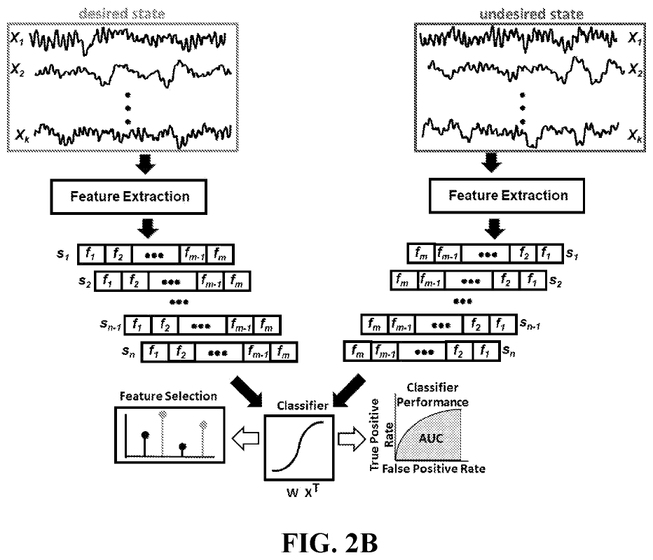

FIGS. 2A-2B illustrate block diagrams of a system for identifying a response biomarker. In this example, of following deep brain stimulation (DBS) is shown in the accompanying figures. As shown in FIG. 2A, the main inputs into the model or framework are the features or biomarkers of corporal states (e.g., body states, body features, electrophysiological features of different brain states, etc.) and their associated labels. The input data is then input into at least one of a classifier and a feature learning module. The primary outputs of the AL framework include classifier performance (e.g., corporal state separability, receiver operating characteristics (ROC), etc.) and one or more identified biomarkers. First, features from desired and undesired corporal state signals may be determined (e.g., calculated). As shown in FIG. 2B, features can be static features (e.g., spectral power) or dynamic features (e.g., state-brain transition probability). In the next step, a binary classification may be utilized to differentiate between desired and undesired corporal states. Biomarkers may be fed to the binary classifier to separate, for example “Stim” from “Non-Stim” samples. A ROC of the cross-validation and an area under the curve (AUC) may be used to measure the classifier performance. Namely, the area under the curve (AUC) is used to assess the separability between Stim and Non-Stim classes. A feature selection method is used to find a subset of features that has the most contribution to this classification. Next, a feature (biomarker) selection method may be used to select a subset of features that have the most contribution to the classification between desired and undesired classes. Any kind of classification model may be utilized. In some instances, the classification model is a logistic regression (LR) model. An LR model classifies desired and undesired samples in the study. One of the main advantages of LR modeling is the interpretability of the models. To this end, any type of explainable artificial intelligence (AI) or interpretable method may be used. This property may be leveraged to identify the important features or biomarkers that discriminate between two classes of data.

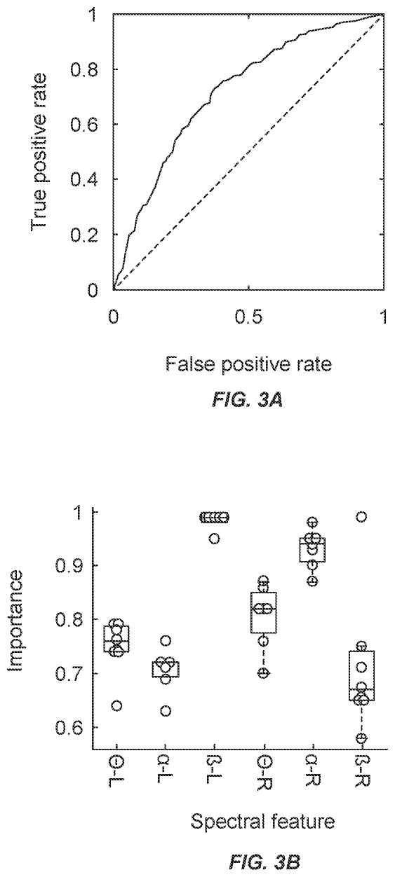

In one specific example, using biomarker identification, a biomarker of the effect of deep brain stimulation (DBS) on the subcallosal cingulate cortex (SCC) in patients with treatment resistance depression (clinical results) may be identified. FIGS. 3A-3B illustrate an example of biomarker identification. FIGS. 3A-3B specifically show acute changes in brain electrophysiology following exposure to intraoperative SCC-DBS. In the specific example shown in FIGS. 3A-3B, anLR model (A) may be used to classify desired and u ndesired samples. To this end, an elastic net regularization (ENR) technique may be utilized. ENR is a regularization and feature selection technique that simultaneously estimates the model parameters and selects the most important neural features by minimizing a cost function, that applies both L1-and L2-regularization. Thus, FIG. 3A illustrates the LR classifier withENR discriminated between baseline (PRE) and post-stimulation (POST) LFPs in the SCC region, where the mean AUC(7)=0.729, and (B) a feature importance score indicating the relative contribution of features to classifier success (PRE v. POST). In this example, β_L is identified as the effect biomarker of SCC DBS.

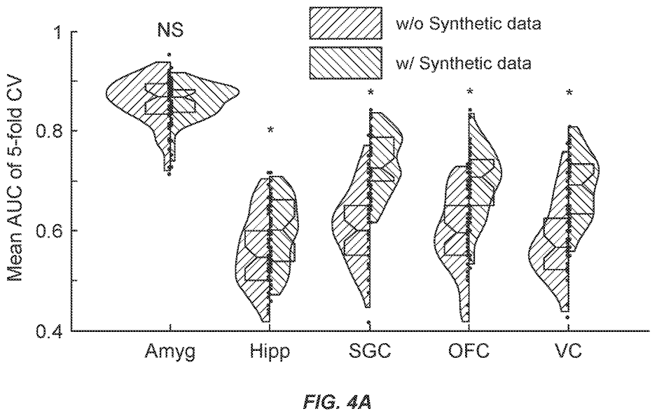

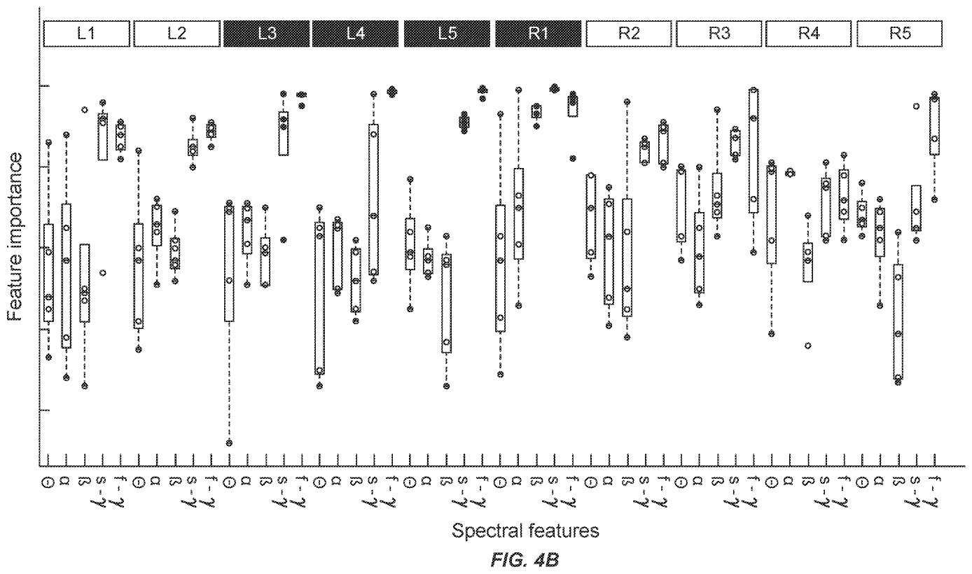

FIGS. 4A and 4B illustrate experimental results from a classification between low symptom state and high symptom state before and after adding synthetic data. In another clinical dataset, a subset of biomarkers were found that separated a high-mood state from a low-mood state in depression. FIG. 4A illustrates that no significant improvement was observed in Amyg, while adding synthetic data improved classification AUC value significantly in Hipp, SGC, OFC, and VC (p<0.0001). FIG. 4B illustrates feature selection results in the subregions of Amyg. Slow-gamma (in L3, L5, and R1), high-gamma (in L3, L4, L4, L5, and R1), and beta power (in R1), are shown in red, contributed more than other features in the classification between two brain states, where p<0.000.

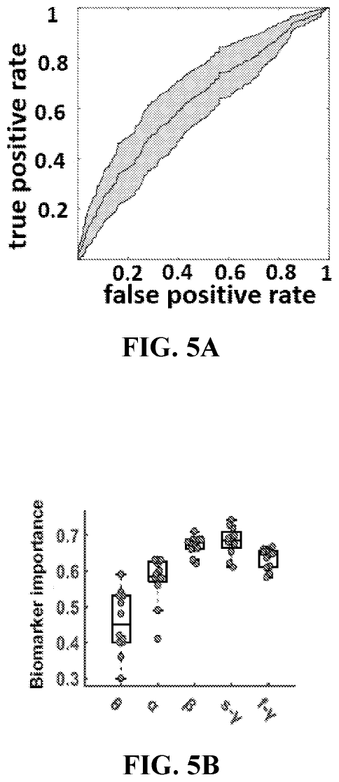

FIGS. 5A and 5B illustrate predicting the memory performance in a one-day test phase based on the CA1 and the amygdala activities during the study phase. In this example, ROC of LR with ENR prediction of “remembered” versus “not-remembered” trials using CAI signal (mean AUC: 0.6561±0.0842, N=12) is shown. As shown in 5B, using biomarker identification, in this example, the biomarker of amygdala stimulation effect on the hippocampal network in a memory enhancement problem is identified. Continuing with the example, s-γ is identified as the amygdala stimulation biomarker. FIG. 5B also illustrates a CA1 biomarker importance graph for classification between “remembered” and “not-remembered” trials combining all subjects with 12-fold nested cross-validation (corrected p<0.01). FIG. 5B further shows that the slow-gamma power in the CA1 contributed most to classification between these two groups. In this example, each dot represents one subject.

The method 100 may also comprise determining 106 a functional link between the biomarker and at least one modulation parameter using, for instance, an active learning framework. After finding the biomarker associated with neurological and neuropsychiatric disorders, the next step is to identify how different stimulation parameters may alter the biomarker. In other words, a regression model may be built between the inputs (i.e., stimulation parameters) and the brain outputs (i.e., target biomarkers) to understand the brain response to the stimulation. To this end, the method may include identifying a functional map between stimulation parameters and brain response. Identifying such a functional map (or regression model) between stimulation parameters and brain response aids in identifying the optimal neuromodulation control strategies. To build this link, a model may be trained from the available data and use that to predict the neural response for untested stimulation parameters. In the passive learning approach, where data is collected through a random sampling process, the learner uses each observation obtained from each sample to update the model parameter. However, no effort is made to choose the best stimulation parameters to improve the learning process more than the other parameters (i.e., optimal parameters). This process usually needs a large amount of training data, which is hard and even impossible to obtain, especially when obtained through expensive and time-consuming clinical experiments.

As described herein, a novel algorithmic approach based on the active learning for optimal data collection and modeling the neurophysiological effects of neuromodulation is disclosed. Active learning, referred to as experimental design in statistics, is a subfield of machine learning and statistics and a smart solution for designing an experiment in which human decision-making is less than optimal for the task. The main rationale underpinning active learning is that data collection is costly, so these query points should be selected in a way such that it optimizes some notion of accuracy for a model being identified. More specifically, active learning is a paradigm in which machine learning models can direct the learning process by providing dynamic suggestions/queries for the “next-best experiment.” The active learning-based framework may be developed for optimal sampling the modulation parameters to characterize the effects of modulation on target biomarkers. This step introduces the development of an algorithmic approach for optimal data collection and modeling the effects of modulation. The active learning-based methods disclosed herein, by which the best model is identified with a limited number of samples, reduces temporal and financial burdens associated with experiments.

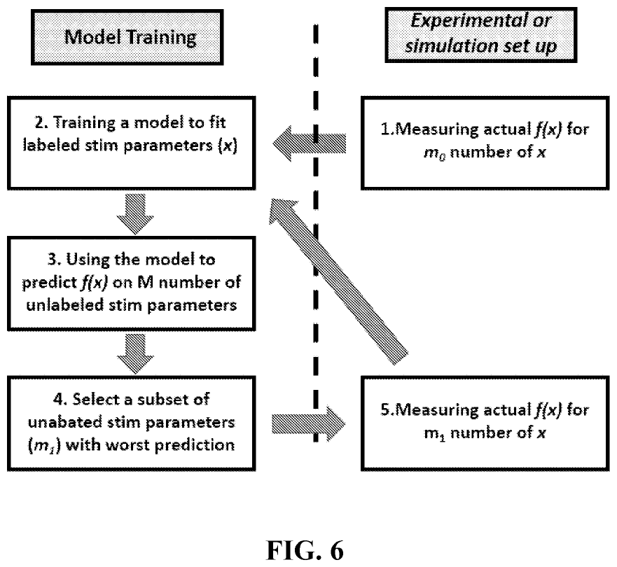

The details of each step are described below, where the diagram of FIG. 6 illustrates the proposed active learning method. The proposed method includes two sides, including model training and an in silico or in vivo environment. The details of the approach are as follows:

In the first step (1), a set, m₀, of modulation parameters may be randomly selected, i.e., x, and measure the response of the respective system to each modulation parameter of m₀. The procedure generates an initial dataset. [symbol] ⁰={(xⁱ, f(xⁱ), i=1, . . . , m₀}.

In a second step (2), using the initial dataset collected in step 1, i.e., [symbol] ⁰, a regression model may be trained to link between the modulation parameter and corporal response.

In a third step (3), based on the trained model, an output of the trained model may be predicted for a very large dataset containing untested modulation parameters.

In a fourth step (4), an m₁ number of modulation parameters may be selected that demonstrate the worst prediction result in step 3 and add them to [symbol] ¹=={(xⁱ, f(xⁱ)), i=1, . . . , m₁}.

In a fifth step (5), through an experimental or simulation setup, the response for the modulation parameter of [symbol] ¹ may be collected.

The process from step (2) to step (5) may be repeated until the prediction of the model and the actual measurements agree with each other. There are two components, including regression modeling and query strategy, that need to be considered when designing an AL framework. A model may be fit between the modulation parameters to the target biomarker. Depending on the non-linearity of the effect of stimulation, linear or nonlinear regression models may be utilized.

After building an initial model based on a selection of random stimulation parameters and their associated brain response, a best stimulation parameter is selected to improve the regression model using a query strategy. The query strategy needs to meet three criteria: informativeness, representativeness, and diversity. The informativeness means that the query strategy should select the stimulation parameters with rich information: collecting the neural response on those parameters would improve the model more than the others. The representativeness of the selected stimulation parameter is evaluated by determining the number of stimulation parameters in proximity to the selected parameter for the subsequent query. By assessing the representativeness of a selected parameter, the method ensures that selected parameters are not outliers. Diversity implies that the selected parameter sets can be distributed across the entire parameter space. By evaluating the diversity of the selected parameter, the method is able to confirm that the selected parameter is not limited to a small localized region within the parameter space.

The method may comprise testing the determined functional link using in-silico modeling. The methodical evaluation of neuromodulation control policies in silico as a preliminary step prior to experimental implementation offers a novel and efficient approach compared to direct in vivo implementation and evaluation by trial and error. Using the procedure and the framework as disclosed herein, active learning may also outperform the random sampling approach in the simulation using the real data from a non-human primate (in silico modeling). The result is reproduceable across multiple sessions.

The method 100 may also comprise applying 108 the functional link may to treat the disorder in the patient. In one aspect, the functional link may be applied by stimulating at least one specific region of the patient's brain. In this manner, the proposed methods for optimizing the neuromodulation control are applied in a real-time in vivo setting.

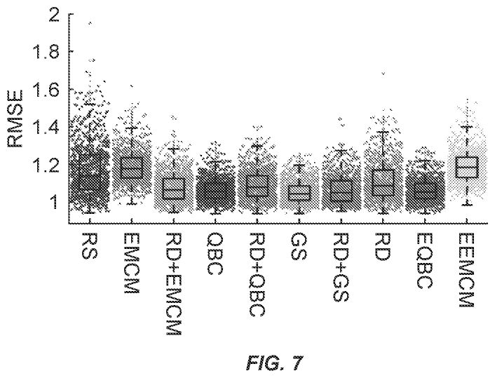

Using the procedure and the framework for identifying the functional link, the active learning framework outperforms the random sampling approach. In the examples shown in FIGS. 7-11, experimental results are shown.

FIG. 7 illustrates an active learning model outperforming the random sampling model. Mean RMSE_AUC of running 1000 times with different AL query strategies and RS sampling. Asterisks represent those AL strategies having significantly less mean AUC_RMSE than RS after multiple corrections (corrected p<0.001).

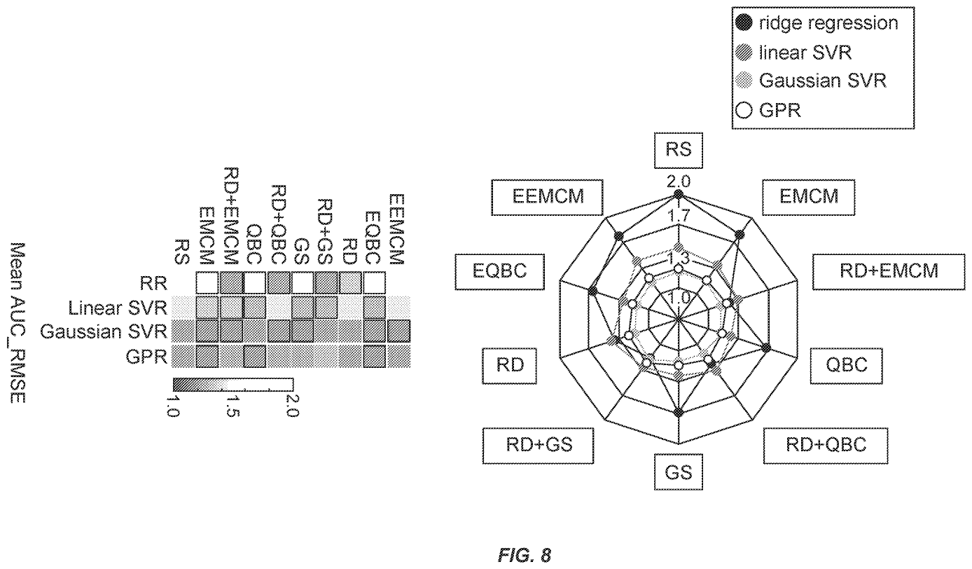

FIG. 8 is a summary plot of models' performance on the Parkinson's disease parameter sweep data. FIG. 8 summarizes the active learning versus random sampling using different regression and query strategies in the synthetic data generated for Parkinson's disease (PD). Mean AUC RMSE of the models on the unseen dataset. AL approach improved both linear and nonlinear models' performance. The most successful approaches were EMCM and EQBC since their model showed less mean AUC_RMSE compared with the RS model in all four regression models. In general, nonlinear models showed less mean AUC_RMSE on the unseen dataset, while the linear model obtained from the RD+GS approach showed the best performance among all models.

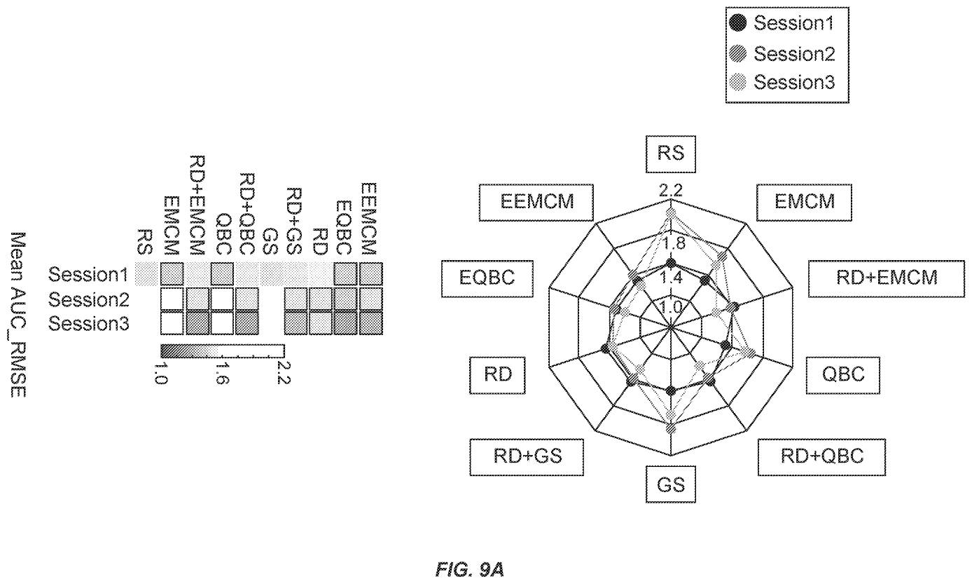

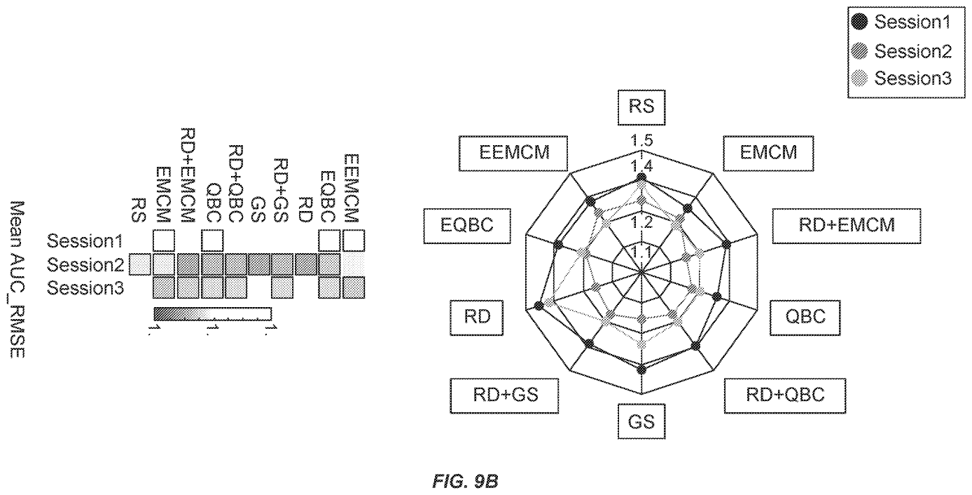

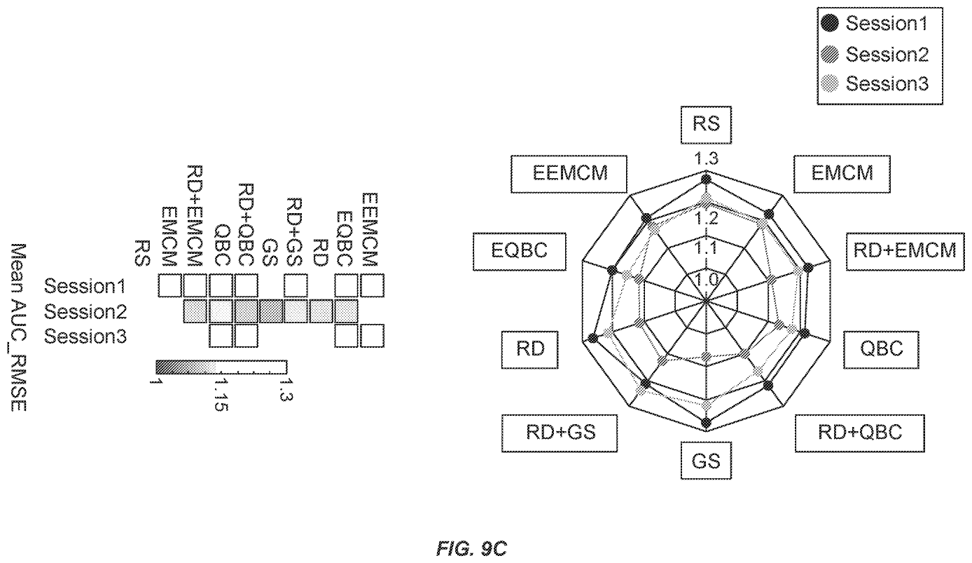

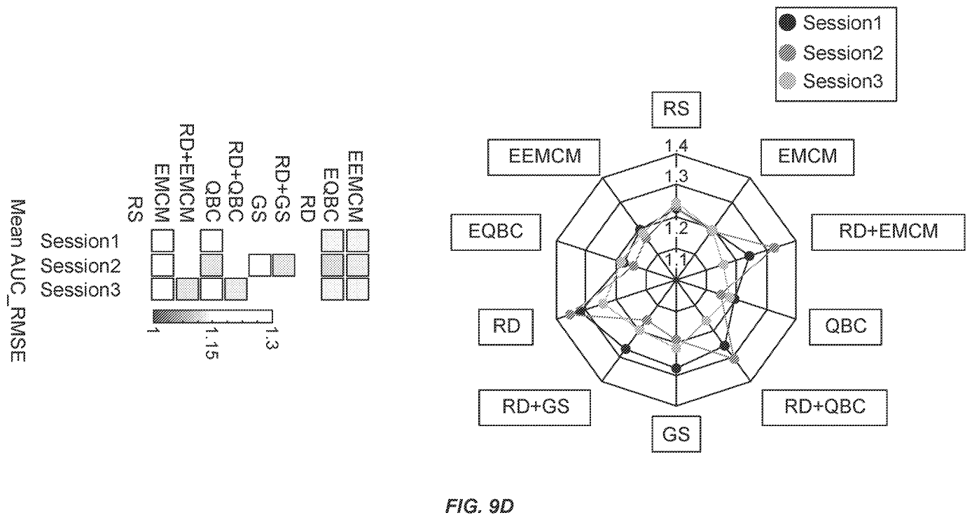

FIG. 9 illustrates an active learning approach outperforming the random sampling approach. Results may be replicated across sessions. FIG. 9 illustrates A) Ridge regression, B) Support vector regression with linear kernel function, C) Support vector regression with Gaussian kernel function, D) Gaussian process regression.

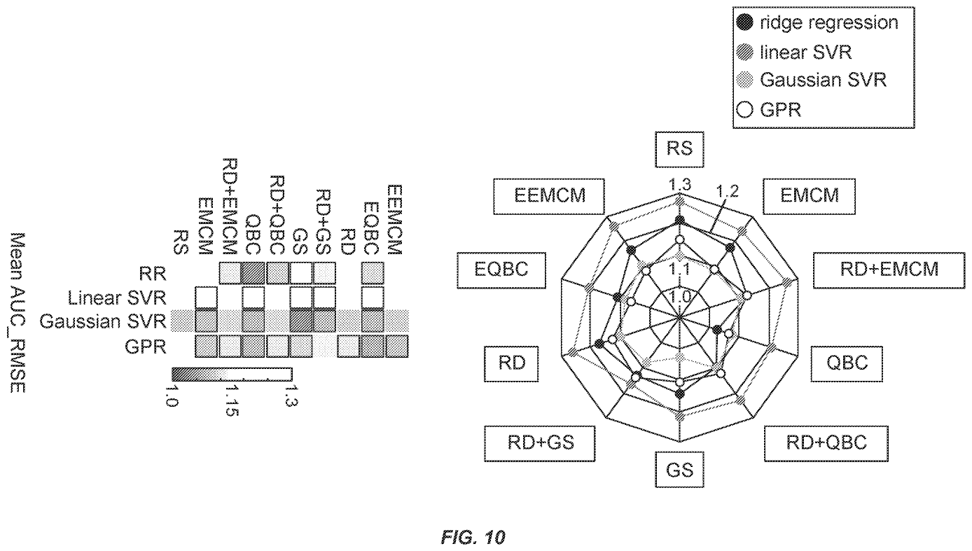

FIG. 10 illustrates performance of the model on the medial septum optogenetic parameter sweep data, further illustrating mean AUC_RMSE of the models on the unseen dataset. AL approach improved both linear and nonlinear models' performance. The most successful approaches were QBC, GS, and EQBC since their model showed less mean AUC RMSE compared with the RS model in all four regression models. In general, nonlinear models showed less mean AUC RMSE on the unseen dataset, while the linear model obtained from the QBC approach showed the best performance among all models. The squares surrounded by a bolded box show significant results compared with the associated RS model performance after multiple comparisons (corrected p<0.001).

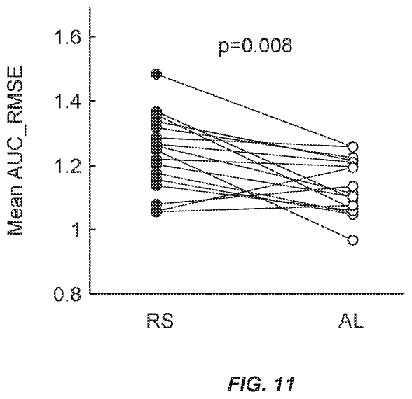

FIG. 11 illustrates AL vs. RS in a real-time in vivo medial septum optogenetic experiment. Using the AL framework described herein, it is shown that the active learning framework outperforms the random sampling approach in the simulation using the real data recorded from a rodent (i.e., in silico modeling). To this end, the method 100 includes a procedure to transfer knowledge from simulation to a real-time experiment. In a real-time experiment on a rat model, the active learning approach is shown to outperform the random sampling one by showing less error on the test data. As can be seen, a ridge regression (RR) model from the query by committee or QBC (green circles) approach outperformed the model from random sampling or RS (blue circles) on 25 unseen test datasets across 20 sessions of the experiment (p=0.008, N=20). As can be seen from the results, RR with QBC shows the best result. Then, an RR model may be used with the QBC query strategy in the real-time experiment.

The methods and systems for treating a patient disclosed herein include three steps. The first step integrates modulation feedback, behavioral information, and artificial intelligence to find a biomarker associated with a specific condition, such as neurological and neuropsychiatric disorders. After finding a biomarker, a functional link is identified between modulation parameters and that biomarker. To this end, an active learning framework is disclosed that optimizes (i.e., minimizes) the number of samples needed to identify this link. In the second step, the link is modeled through an in-silico modeling approach. This step may be performed prior to running an in vivo and real-time experiment. Finally, in the third step, an in vivo experiment may be conducted using the prior knowledge obtained from the step of identifying the functional link.

The present systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

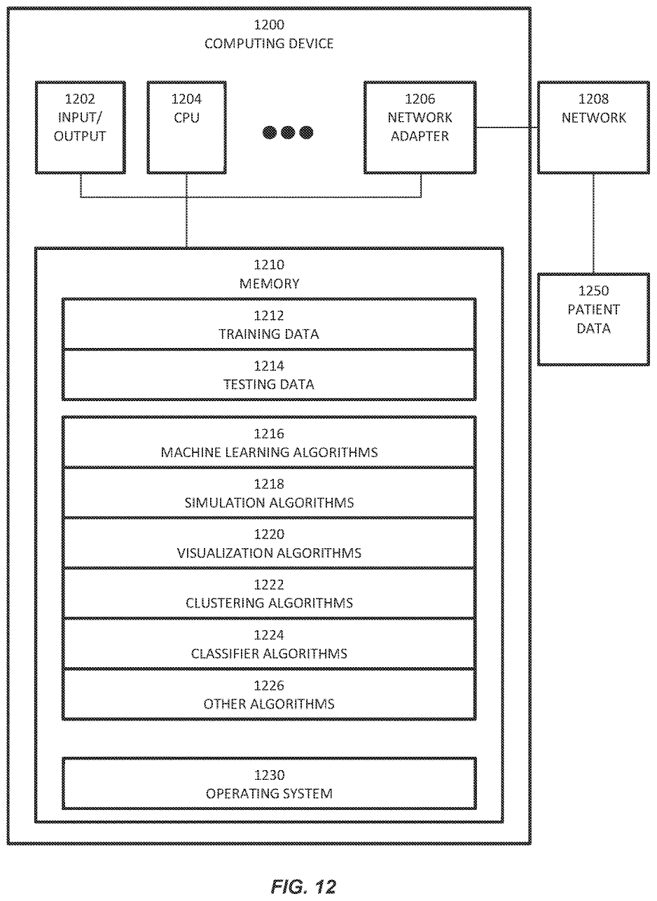

FIG. 12 illustrates a computing device 1200 as it relates to the present disclosure. The computing device 1200 may, for example, perform calculations, execute routines and algorithms, process data, communicate with other devices via a network, and display results. For example, a computing device 1200 may comprise a processor or CPU 1204, a network adapter 1206 for communication with a network 1208. The network 1208 may connect the computing device 1200 to external data sources such as patient data 1250 or to other computers (not shown in the figure). The computing device may comprise an input/output device 1202. Such an input/output component 1202 may be an input device, an output device, or both and the computing device 1200 may have several such components. Example input devices 1202 include a keyboard, a mouse, a microphone, a touchpad, a joystick, and the like. Example output devices 1202 include a display, a speaker, a haptic feedback device, and the like. The computing device 1200 may further comprise memory 1210 or a computer readable storage medium 1210. In the computer memory 1210 may reside instructions for carrying out the methods and techniques described elsewhere in this disclosure. The computer memory 1210 may also comprise an operating system 1230 for control of the various parts and components of the computing device 1200. The memory 1210 may also store data, for example training data 1212 and testing data 1214, or other data (not shown in the figure). The memory 1210 may also comprise algorithms such as machine learning algorithms 1216, simulation algorithms 1218, visualization algorithms 1220, clustering algorithms 1222, classifier algorithms 1224, or other algorithms 1226.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network 1208, for example, the Internet, a local area network, a wide area network and/or a wireless network. The network 1208 may comprise copper transmission cables, optical transmission fibers, wireless transmission, routers, firewalls, switches, gateway computers, and/or edge servers. A network adapter card 1206 or network interface in each computing/processing device 1200 receives computer readable program instructions from the network 1208 and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for treating a patient comprising the steps of:
receiving a set of information describing the patient;
identifying a biomarker associated with a condition affecting the patient;
determining a functional link between the biomarker and at least one modulation parameter by using an active learning framework; and
applying the functional link to treat the condition in the patient by modulating at least one specific region of the patient.

2. The method of claim 1, wherein the identifying step comprises:
training a classifier to identify a plurality of biomarkers that differentiate corporal states as desired or undesired, or as in a pre-treatment or a post-treatment state.

3. The method of claim 2, wherein the plurality of biomarkers are static biomarkers, dynamic biomarkers, or both.

4. The method of claim 2, wherein the determining step comprises:
identifying a relative contribution of two or more biomarkers of the plurality of biomarkers to identify the corporal state as desired or undesired; and
identifying at least one biomarker as having a highest relative contribution to identifying the corporal state as desired or undesired.

5. The method of claim 4, wherein the determining step comprises:
using a feature learning technique along with the identified at least one biomarker to select a set of most important biomarkers and estimate one or more optimal model parameters.

6. The method of claim 5, wherein the feature learning technique is an elastic net regularization technique.

7. The method of claim 1, wherein the determining step comprises:
determining a regression model between the at least one modulation parameter and the identified biomarker.

8. The method of claim 1, further comprising, prior to the applying step, testing the determined functional link using in-silico modeling.

9. A system for treating a patient comprising a processor and memory, wherein computer instructions are stored in the memory to cause the processor to perform the steps of:
receiving a set of information describing the patient;
identifying a biomarker associated with a condition affecting the patient;
determining a functional link between the biomarker and at least one modulation parameter by using an active learning framework; and
applying the functional link to treat the condition in the patient by modulating at least one specific region of the patient.

10. The system of claim 9, wherein the identifying step comprises:
training a classifier to identify a plurality of biomarkers that differentiate corporal states as desired or undesired, or as in a pre-treatment or a post-treatment state.

11. The system of claim 10, wherein the plurality of biomarkers are static biomarkers, dynamic biomarkers, or both.

12. The system of claim 10, wherein the determining step comprises:
identifying a relative contribution of two or more biomarkers of the plurality of biomarkers to identify the corporal state as desired or undesired; and
identifying at least one biomarker as having a highest relative contribution to identifying the corporal state as desired or undesired.

13. The system of claim 12, wherein the determining step comprises:
using a feature learning technique along with the identified at least one biomarker to select a set of most important biomarkers and estimate one or more optimal model parameters.

14. The system of claim 13, wherein the feature learning technique is an elastic net regularization technique.

15. The system of claim 9, wherein the determining step comprises:
determining a regression model between the at least one modulation parameter and the identified biomarker.

16. The system of claim 9, further comprising, prior to the applying step, testing the determined functional link using in-silico modeling.

17. A computer-readable storage medium storing instructions, an execution of which in a computer system causes the computer system to perform operations comprising:
receiving a set of information describing the patient;
identifying a biomarker associated with a condition affecting the patient;
determining a functional link between the biomarker and at least one modulation parameter by using an active learning framework; and
applying the functional link to treat the condition in the patient by modulating at least one specific region of the patient.

18. The computer-readable storage medium of claim 17, wherein the identifying step comprises:
training a classifier to identify a plurality of biomarkers that differentiate corporal states as desired or undesired, or as in a pre-treatment or a post-treatment state.

19. The computer-readable storage medium of claim 18, wherein the determining step comprises:
identifying a relative contribution of two or more biomarkers of the plurality of biomarkers to identify the corporal state as desired or undesired; and
identifying at least one biomarker as having a highest relative contribution to identifying the corporal state as desired or undesired.

20. The computer-readable storage medium of claim 19, wherein the determining step comprises:
using a feature learning technique along with the identified at least one biomarker to select a set of most important biomarkers and estimate one or more optimal model parameters.
